(19) United States
(12) Patent Application Publication
(10) Pub. No.: US 2006/0084134 A1
(43) Pub. Date: Apr. 20, 2006

(54) Direct ethanol metabolite ethyl sulfate as an useful diagnostic and therapeutic marker of alcohol consumption

(76) Inventor: Friedrich Martin Wurst, Oberwil (CH)

Correspondence Address: JOYCE VON NATZMER, 4615 NORTH PARK AVENUE, SUITE 919, CHEVY CHASE, MD, 20815, US

(21) Appl. No.: 11/149,427

(22) Filed: Jun. 10, 2005

Related U.S. Application Data
(60) Provisional application No. 60/578,314, filed on Jun. 10, 2004.

(51) Int. Cl. C12Q 1/26

(52) U.S. Cl. 435/25

(57) ABSTRACT

Accurate self report strategies, biological state markers, combinations of alternative biomarkers, and combinations of biomarkers and self reports capable of monitoring alcohol consumption with a high sensitivity and specificity over a broad time spectrum are disclosed.

In particular, the use of ethyl sulfate (monoethylsulfate, molecular weight 126 g/mol, C₂H₅SO₄H) to elucidate ethanol intake is described in the context of screening monitoring in various settings, e.g. a) after liver transplantation b) methadone maintenance patients with hepatitis C and comorbid excessive alcohol use c) underage drinking d) rehabilitation programs for alcoholics motivational feedback to improve knowledge on drinking patterns differentiating moderate/social drinking from problematic/harmful drinking differential diagnosis (e.g. elevated transaminases) evaluating treatment programs and drug trials elucidating the role of neuropsychological impairment following alcoholisation (i.e. hangover state) which plays a major role in accidents, disclosing recent drinking in social drinkers in risky situations (driving, workplaces, pregnancy (fetal alcohol syndrome (FAS)), and general monitoring.

### CROSS-REFERENCE TO RELATED APPLICATIONS

This application claims the benefit of U.S. provisional application No. 60/578,314, filed Jun. 10, 2004, the entire content of which is incorporated herein by reference.

### BACKGROUND OF THE INVENTION

1. Field of the Invention

The invention is directed at methods for detecting and/or quantifying the ethanol intake in humans and/or non-human animals.

2. Related Art and Introduction

The global burden of alcohol induced diseases exceeds those induced by tobacco and is on a par with the burden resulting from unsafe sex practices world-wide (WHO, Global Status Report on Alcohol, 1999). The currently used indirect biological state markers of alcohol intake [symbol] showshortcomings that limit their value. These include:
the time spectrum of detection they reflect (e.g. serum ethanol detects only recent use within hours);
influences of age, gender and a variety of substances and non-alcohol-associated diseases (Gilg and Soyka, 1997; Laposata, 1999).

Those markers are disadvantageously also influenced by body mass index (BMI) and medication thereby limiting their value. In contrast, direct ethanol metabolites, such as fatty acid ethyl esters (FAEE), ethyl glucuronide (EtG) and phosphatidylethanol (PEth) seem to lack many of those shortcomings.

Each of those remains positive in serum and urine for a characteristic time spectrum after the cessation of ethanol intake—FAEEs is detectable in serum up to 24 h, EtG in urine up to 5 days, PEth in whole blood more than to 2 weeks. Additionally, EtG and FAEE can be detected in hair for months.

During the last decade significant contributions were made to the clinical/forensic applications of EtG, FAEE and PEth as well as to a basic characterization of EtG, and the clinical dimensions of EtG, FAEE and PEth such as excretion characteristics, distribution in various body fluids, tissues etc, monitoring of forensic psychiatric patients and physicians recovering from addiction, giving motivational feedback to rehabilitating alcohol dependent patients etc. A variety of techniques for determination of EtG, (gas chromatography-mass spectrometry (GC/MS), liquid chromatography-tandem mass spectrometry (LC-tandem MS) [very recently with two ion transitions monitored, according to forensic standards], using deuterium labeled EtG as internal standard [a gold standard] etc.) have been developed.

The studies completed so far—which cumulatively consist of more than 4500 urine and serum samples of more than 1700 individuals—include participation in the World Health Organisation/International Society for Biomedical Research on Alcoholism (WHO/ISBRA) study and the very first comparisons of this new line of markers among each other but also in comparison to traditional markers and self reports (EtG, FAEE, PEth, gamma glutamyl transpeptidase (GGT), mean corpuscular volume (MCV), carbohydrate deficient transferring (CDT), hydroxytryptophol/hydroxyindolic acid (HTOL/HIAA) ratio, glucuronated HTOL (GTOL); breath and urinary ethanol, EtG, PEth, CDT, MCV, GGT). Recent work also focuses on evaluation of aspects of leptin as a putative state, trait and craving marker in correlation to previous ethanol intake as measured by biomarkers and self reports.

Throughout this specification reference is made to certain publications including patent publications which also appear in the list of references appended hereto. All of these publications referred to throughout the specification and listed in appended the list of references are incorporated herein by reference in their entirety.

Especially during the last decade, three non-oxidative direct ethanol metabolites have attracted attention. Promising markers include:
fatty acid ethyl esters (FAEEs) (Wada et al., 1971, Dan and Laposata, 1997, Diczfalusy et al., 1999; Diczfalusy et al., 2001, Wurst et al., 2003b, 2004),
ethyl glucuronide (EtG) (Alt et al., 2000; Dahl et al., 2002; Schmitt et al., 1995; Seidl et al., 2001; Wurst et al. 1999a, b, 2000, 2002, 2003a, b), and
phosphatidyl ethanol (PEth) (Ailing et al., 1983, 1984; Gunnarson et al., 1998; Hansson et al., 1997; Varga et al., 1998,2000; Wurst et al., 2003a, 2004).

Each of these remains positive in serum and urine for a characteristic time spectrum after the cessation of ethanol intake—FAEEs in serum up to 24 h, EtG in urine up to 5 days, PEth in whole blood more than to 2 weeks. Additionally, EtG and FAEE can be detected in hair for months (Alt et al., 2000; Wurst et al, 2003b,2004).

Ethyl Glucuronide

To help fill the gap on the time axis with regard to recent alcohol consumption occurring between hours to 1 week, a state marker capable of monitoring alcohol consumption within this intermediate period is desirable.

Although, Neubauer described in 1901 in Prague the detoxifying pathway of alcohol elimination via conjugation with activated glucuronic acid, it was not until the 1990s, that this minor metabolite of ethanol aroused greater interest. Ethyl glucuronide (EtG), is a non-volatile, water-soluble, stable, direct metabolite of ethanol. The molecular formula of EtG is C₈ H₁₄ O₇, the molecular weight is 222 g/mol and the melting point (decomposition temperature) is about 150° C. Shortly after the consumption of even minor amounts of alcohol like 10 grams (half a bottle of beer)(Weinmann et al., 2004), EtG becomes positive and is dose-dependently detectable for 4 days following complete elimination of alcohol from the body (Weinmann et al., 2004; Wurst et al., 1995, 1996, 1999a-c, 2000a-b, 2001, 2002a,b, 2003a,b). With its specific time frame of detection, intermediate between short- and long-term markers and with its high sensitivity and specificity, ethyl glucuronide is a promising marker of alcohol consumption in general and a marker for lapse and relapse control.

Wurst and colleagues have contributed to the characterization of EtG in basic and in clinical dimensions such as
excretion characteristics (Wurst et al., 1995, 1996, 1999a, 2002a),
distribution in various body fluids, tissues etc (Wurst et al, 1999b),
elucidation of the usefulness of EtG in comparison to other biological state markers and demonstration of the robustness of the LC/MS-MS (Liquid Chromatography/Mass Spectrometry/Mass Spectrometry) method in large numbers as part of the WHO/ISBRA Study on State and Trait Markers of Alcohol Use and Dependence (Wurst et al., 2002b, 2004 (in press))
through monitoring of forensic psychiatric patients (Wurst et al., 1999a,2000a,2003a) and
of physicians recovering from addiction (Wurst et al., 2004, in press),
through providing motivational feedback to rehabilitating alcohol dependent patients (Wurst et al., 1999a, 2003b) etc.

A variety of techniques for determination of EtG, (GC/MS, LC-tandem MS [recently even with two ion transitions monitored, according to forensic standards (Weinmann et al., 2004)], using deuterium labeled EtG (d₅-EtG) as internal standard [a gold standard] etc.) have been developed:

| | |
|---|---|
| GC/MS | Alt et al., 1999 |
| LC/MS-MS | Wurst et al., 1999a-c, 2000a-c, 2002, |
| | Weinmann et al., 2004 |

The studies that have been done, which cumulatively consist of more than 4500 urine and serum samples of more than 1700 individuals, include those from participation in the WHO/ISBRA study, the very first comparisons of this new line of markers among each other, but also in comparison to traditional markers and self reports (for overview, see Wurst et al., 2003b).

The cumulative findings emphasize, that altogether, the test for ethyl glucuronide can be used for
screening
monitoring
motivational feedback
to improve knowledge on drinking patterns
differentiate moderate/social drinking from problematic/harmful drinking
differential diagnosis (e.g. elevated transaminases)
evaluate treatment programs and drug trials
elucidate the role of neuropsychological impairment following alcoholisation (i.e. hangover state) which plays a major role in accidents,
disclose recent drinking in social drinkers in risky situations (driving, workplaces, pregnancy (FAS)).

Phosphatidylethanol

Phosphatidyl ethanol (PEth) is formed in mammalian cells when ethanol is present (Alling et al., 1983). Studies have supported use of phosphatidyl ethanol (PEth) in blood as a marker of alcohol abuse (for review, see Varga et al., 2001). A daily intake of about 50-60 g of ethanol for 2-3 weeks (1000 g cumulatively) are needed for PEth to be positive (>LOD (limit of determination)) in blood.

In two recently published studies—each employing a considerable marker battery (EtG, PEth, FAEE in hair, CDT, MCV, GGT, breath and urinary ethanol)—Wurst et al. could demonstrate that for known alcoholic patients, there were
no false positives (Wurst et al., 2003a) and
no false negatives (Wurst et al., 2004) for sober subjects for PEth, thereby indicating extraordinary high sensitivity and specificity.

Fatty Acid Ethyl Esters

Fatty acid esters of ethanol (FAEEs) have been implicated as possible mediators for at least some of the toxic effects associated with alcohol consumption and serve well as short term markers of ethanol intake in serum and long term markers in hair. In serum and erythrocytes, FAEEs can be detected up to 24 h after drinking. They are not stable in blood samples due to continued enzyme activity, but are incorporated into hair and can be analyzed in that medium even after several months (Pragst et al. 2001). In a variety of clinical and forensic situations long term use of alcohol must be monitored.

In a recently published project Wurst et al. (2004) explored the utility of fatty acid ethyl esters (FAEE) in this regard. The sum of the concentrations (C_{FAEE}) of the four—most prominent—esters, ethyl myristate, ethyl palmitate, ethyl oleate and ethyl stearate was used as an alcohol marker. Drinking validation criteria include self reports, phosphatidyl ethanol (PEth) in whole blood as well as the traditional markers of heavy drinking, gamma glutamyl transpeptidase (GGT) mean corpuscular volume (MCV) and carbohydrate deficient transferrin (CDT).

The figure is the first to compare the mentioned markers, taken from a single patient during detoxification [BAC: Blood alcohol concentration; SEtG: EtG concentration in serum; UEtG: EtG concentration in urine; FAEE: fatty acid ethyl esters in serum. The patient was admitted at −11 hours with a BAC of 1.99 g/kg; O is the time point when BAC was <limit of determination (LOD) (Wurst et al, 2003b)

FAEE levels from 18 alcohol dependent patients in detoxification were contrasted with those of 10 social drinkers and 10 teetotalers. FAEEs in hair were determined, using headspace solid phase microextraction and gas chromatography mass spectrometry. C_{FAEE}, as sum of the concentrations of four esters, was compared to a major FAEE, ethyl palmitate. PEth was measured in heparinized whole blood with a high pressure liquid chromatography (HPLC) method.

ROC curve analysis for C_{FAEE}, indicated a sensitivity of 100% and a specificity of 90% for a cut off of 0.29 ng/mg. By using a cut-off of 0.4 ng/mg, C_{FAEE} identified 94.4% correctly. C_{FAEE} and ethyl palmitate were significantly associated (r=0.945, p<0.001) as were C_{FAEE} and PEth (r=0.527, p=0.025). By contrast, among the serum and blood markers, % CDT identified 47.1%, MCV 38.8% and GGT 72.2% of patients with chronic intake of higher amounts of ethanol correctly, whereas PEth achieved 100% accuracy.

The data suggest that C_{FAEE} is a potentially very valuable marker of chronic intake of high quantities of ethanol. Furthermore, the results indicate that a reasonable FAEE cut off to distinguish between social/moderate and heavy drinking/alcoholism is 0.4 ng/mg.

Granted their ability to mark heavy drinking use over long periods of time, FAEE hair levels may play an important and unique role in both clinical practice and research.

Despite the described developments in the field of direct ethanol metabolites for evaluating alcohol consumption, there remains a need for alternative direct ethanol metabolites to improve self-report strategies, provide biological state markers, provide combinations of biomarkers, including new biomarkers, for monitoring alcohol consumption, and combinations of biomarkers and self reports to monitor alcohol consumption with high sensitivity and specificity over a wide time spectrum as required for different applications.

Direct ethanol metabolites, in particular ethyl sulfate (monoethylsulfate, molecular weight 126 g/mol, C₂H₅SO₄H), have good potential for close to ideal biomarkers of ethanol intake. Methods for monitoring ethyl sulfate to elucidate ethanol intake are described herein. The development of this use is based, at least in part, on the insight, that determination, e.g. with an LC-MS-MS method, should be similar to EtG. The person skilled in the art will appreciate that many materials and methods used in the context of the ethanol metabolites described above can also be used, with appropriate modifications, in the context of the applications of EtS (ethyl sulfate) described herein.

Definitions

“A body sample” in the context of the present invention includes, but is not limited to, all body fluids (such as blood, serum, plasma, urine, cerebrospinal fluid, synovial fluid, saliva etc), all tissues (liver, kidney, brain, fat, etc), and all other components of the body in humans and animals (meconium, faeces, hair, nail etc).

“Obtaining a sample” in the context of the present invention includes both in vitro and in vivo sampling.

### SUMMARY OF THE INVENTION

Some, though not all, uses and applications of EtS that are within the scope of the present invention, are
screening
monitoring in various settings, e.g.
   after liver transplantation
methadone maintenance patients with hepatitis C and comorbid excessive alcohol use
   underage drinking
   rehabilitation programs for alcoholics
motivational feedback
to improve knowledge on drinking patterns
differentiate moderate/social drinking from problematic/harmful drinking
differential diagnosis (e.g. elevated transaminases)
evaluate treatment programs and drug trials
elucidate the role of neuropsychological impairment following alcoholisation (i.e. hangover state) which plays a major role in accidents
disclose recent drinking in social drinkers in risky situations (driving, workplaces, pregnancy (fetal alcohol syndrome (FAS)), and
elucidating evolutionary aspects such as ethanol intake in primates.

EtS uses that are within the scope of the present invention are also:
Diagnostic (such as screening, monitoring etc) and therapeutic (such as motivational feedback etc) use of EtS for assessing ethanol intake in use, abuse and dependence; (This includes the use as confirmatory test, following e.g. a positive EtG testing)
The combination of EtS with other markers, including ratios, e.g., of EtS and EtG;
in all body fluids (such as blood, serum, plasma, urine, cerebrospinal fluid, synovial fluid etc), all tissues (liver, kidney, brain, fat, etc), and all other components of the body in humans and animals (meconium, faeces, hair, nail etc);
testing food, food-like substances, beverages and medications for the presence of EtS.
   by means of
1) instrumental analysis (GC/MS, LC/MS, LC/MS-MS, high pressure liquid chromatography (HPLC), magnetic resonance technology (NMR), Densitometry, Spectroscopy, Thinlayer chroatography (TLC), capillary electophoresis (CE), Transdermal Applications),
2) antibody based (mono- and polyclonal) determination techniques (enzyme linked immuno sorbent assay (ELISA), radio immuno assay (RIA), fluorescence based),
3) standards for EtS: deuterium labeled EtS, radio labeled EtS etc.,
4) use of nano technology for EtS determination and production of means for determination,
5) fluorescence polarisation in homogenous assay(s),
5) applications (see above) and forms of application. This might include using ratios of EtS concentration in serum and urine, ratios of EtG and EtS, including the activity of enzymes that play a role in the formation and degradation of EtS.

In one embodiment, the present invention is directed to a method for detecting and/or quantifying ethanol intake in a human and/or non-human animal comprising:
obtaining a body sample from said human and/or non-human animal,
determining a presence or level of EtS in said body sample, and
correlating said presence or level of EtS in said body sample to the ethanol intake of said human and/or non-human animal.

The presence or level of EtS in said body sample may be determined via methods inclduing, but not limited to, GC/MS, LC/MS, LC/MS-MS, high pressure liquid chromatography (HPLC), magnetic resonance technology (NMR), Densitometry, Spectroscopy, thin layer chromatography (TLC), capillary electophoresis (CE), transdermal application(s) or fluoresence polarisation.

In another embodiment of the present invention, the presence or level of EtS in said body sample is determined by one or more antibody based (mono- and polyclonal) determination technique. Said antibody based determination technique includes, but is not limited to, enzyme linked immuno sorbent assay (ELISA), radio immuno assay (RIA) or a fluorescence based assay.

In yet another embodiment of the present invention, the ethanol intake is quantified using deuterium labeled EtS or radio labeled EtS.

In yet another embodiment of the present invention, EtS is detected via nanotechnogical methods, including, but not limited to, nanotechnological methods as those used in the context of other markers, such as SDT.

The invention is also directed to screening and/or monitoring the intake of ethanol in a human via the method described herein.

The method of the present invention may be performed, 1 to 24 hours, 1 to 7 days, weeks, or months after ethanol intake.

In another embodiment the method of the present invention is combined with at least one second method for determining ethanol intake in a human. Said second method may be, but is not limited to, ethyl glucuronide testing, phosphatidylethanol testing or fatty acid ethyl ester testing and wherein said second method is performed prior and/or after said method of the present invention.

According to one aspect of the invention, the presence or level of EtS is measured in more than one type of sample.

According to one embodiment of this aspect the ratio of EtS in serum/urine is determined.

According to another aspect of the present invention, the presence or level of EtS is determined indirectly (e.g. via acid hydrolysis or the enzymatic activity of sulfatase).

In another aspect of the present invention, the presence or level of EtS is determined indirectly via one or more enzymes that is involved in the formation or degradation of EtS.

### BRIEF DESCRIPTION OF THE DRAWINGS

FIG. 1 shows ion chromatograms of ethylsulfate sodium salt obtained from ABCR Karlsruhe in Germany.

FIG. 2 depicts ion chromatograms of urine sample of drinking experiment (0.5 L red wine) acquired in Multiple-Reaction Monitoring Mode showing traces of the indicated specific mass-spectrometric transitions with EtS at 3.99 min and EtG/D5-EtG between 4.5 and 5.2 min retention time.

FIG. 3 shows an urinary excretion profile of ethanol (urine alcohol concentration, UAC), EtG and EtS, and normalized to creatinine (100 mg/dL) EtG-100 and EtS-100.

### DETAILED DESCRIPTION OF VARIOUS AND PREFERRED EMBODIMENTS

Monoethylsulfat

Ethyl sulfate (EtS) is—like EtG, FAEEs and PEth—a direct ethanol metabolite, presumably formed by sulfotransferases. The molecular weight of EtS is 126 g/mol, the formula C₂H₅SO₄H. A solution of EtS in ethanol and sulfuric acid has the nomen officinalis “mixtura sulfurica acida seu Acidum Halleri.” EtS is commercially available as sodium salt. Enzymatic breakdown of EtS is via sulfatase. The formation of ethylsulfate by conjugation of activated sulfate and ethanol by rat liver has already been reported in 1959 by Vestermark and Boström, and the detection in rat urine after application of ³⁵S-sulfate and ethanol to rats was performed by thin-layer chromatography and by autoradiographic detection on X-ray films (Boström and Vestermark, 1960). Lung tissue was found to also have the ability to metabolize ethanol via glucuronidation [Bernstein et al., 1983) and by sulfation (Bernstein et al., 1984, 1990). Later, Manautou and Carlson (1992) compared the hepatic and pulmonary metabolism via glucuronidation and sulfation in rats and rabbits. Meanwhile, for sulfate conjugation a superfamily of cytosolic sulfotransferases has been described and is showing a genetic polymorphism (Carlini et al., 2001). The conjugation of aliphatic alcohols in human has been mentioned by Bonte et al., 1981, when investigating metabolites of higher aliphatic alcohols; detection has been performed indirectly via cleavage by sulfatase and analysis of the alcohols. However, no direct analytical method has been available until very recently, to detect and quantify EtS as a marker for recent ethanol consumption in humans. In parallel to our work, Beck and Helander, 2004 have developed an LC-ESI-MS assay using single-quadrupole mode and D₅-ethylglucuronide as internal standard for quantitation of EtS in urine samples. However, selected ion monitoring of the deprotonated molecule with a single-stage quadrupole MS must not be applied to forensic cases as the only method for compound detection—since it does not fulfill forensic criteria to prove compound identity (Aderjan et al., 2000).

Finally, the potential of ethyl sulfate (monoethylsulfate) to elucidate ethanol intake was discovered based on the insight of the similarities between EtG and EtS by Friedrich Martin Wurst and in the very recent months has been brought to application, based on the hypothesis, that determination e.g. with an LC-MS-MS method should be similar to that used for EtG (Weinmann et al., 2004). The present invention is directed at detecting and/or quantifying the intake of ethanol over a wide time span, including minutes, hours, days, weeks and months after intake. It is understood that at any point in time during this time span such a detection and/or quantification can be performed. The person skilled in the art will understand which of the disclosed methods can be used at any given point in time.

Material and Methods

Ethyl sulfuric acid sodium salt (C₂H₅NaO₄S), CAS: 546-74-7, as referred to herein was provided by ABCR GmbH & Co. KG (Karlsruhe, Germany).

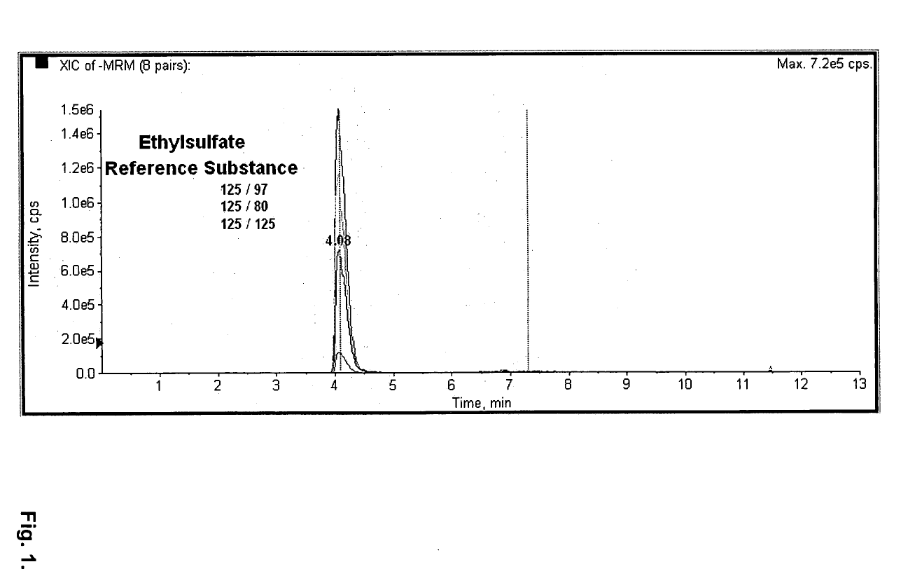

Characteristic tandem-mass spectrometric fragmention of EtS, purchased from ABCR Karlsruhe/Germany, was obtained when analyzing EtS solution in product ion scan mode: deprotonated molecule (M-H)⁻, m/z 125 and fragment ions m/z 80 and 97. An LC-MS/MS-method, which has previously been published for the determination of EtG (Weinmann 2004), was modified by inclusion of the transitions for EtS. Subsequent LC/MS/MS-analysis was performed in MRM-mode (m/z 125 to m/z 97 and m/z 80 and by monitoring the unfragmented precursor ion (m/z 125) of EtS (ion chromatograms of diluted reference solution see FIG. 1). A gradient elution with increasing acetonitrile concentration of the eluting solvent was used—all other chromatographic equipment was as described in the article by Weinmann et al. (Weinmann 2004).

Mass Spectrometric Data for Structural Characterization

Mass spectrometric data for structural characterization using negative mode LC-ESI-MS/MS with triple-quadrupole instrument:
[M-H]⁻ 125, fragment ions (SO3⁻, m/z 80; HSO4⁻: m/z 97)
Retention time: 3.99 min.

Controlled Drinking Experiment with a Healthy Volunteer

Urine Samples:

Urine samples were obtained from a volunteer after drinking 0.54 L of wine (12 vol %, 49 g ethanol). Sample preparation was done by addition of D5-EtG and dilution of the urine sample prior to injection in the LC-MS/MS system. Quantification was performed using D5-EtG as internal standard.

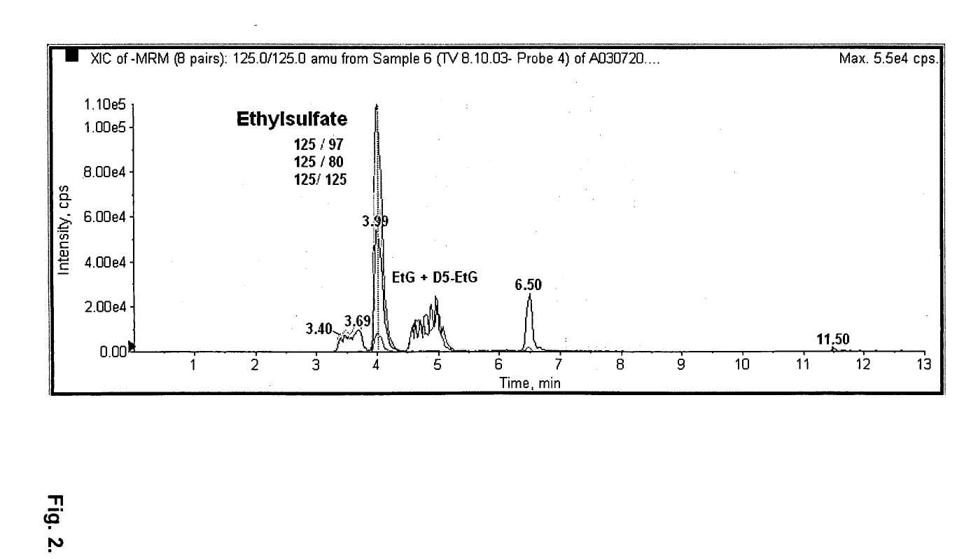

FIG. 2 shows the analysis of a urine sample (retention time EtS-ca. 3.99 min; EtG at 5 min—the method was not optimized for EtG and D5-EtG)

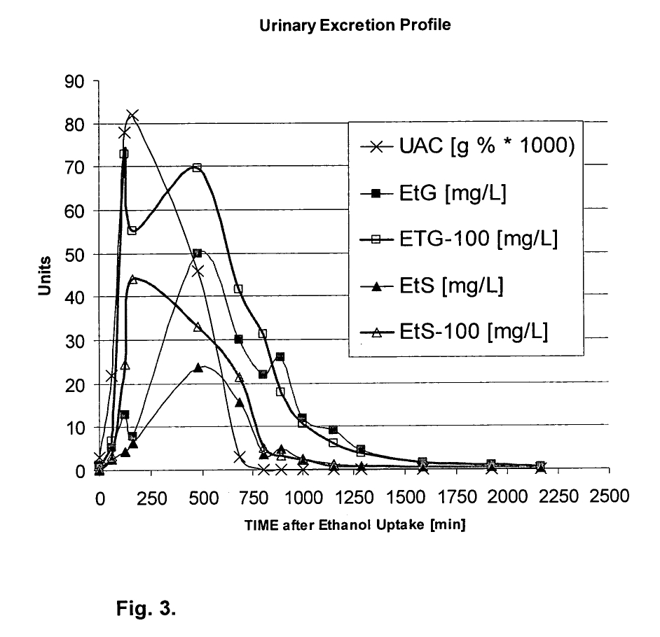

In this first drinking experiment with a healthy male volunteer (540 ml of wine, 12 vol %, 49 g ethanol), EtS was detectable in urine for up to 36 hours (FIG. 3). As expected, also EtG still was positive in urine. However, at this time point, urinary ethanol (UAC) had not been detectable since 23 hours. FIG. 3 and table 1 show the excretion of EtS and EtG in urine.

TABLE 1
Drinking experiment with 540 ml of wine: urinary excretion profile of ethanol
(urine alcohol concentration, UAC), EtG and EtS, and normalized to creatinine
(100 mg/dL) EtG-100 and EtS-100 (see FIG. 3).

| Time after | Time after | | | | | | |
|---|---|---|---|---|---|---|---|
| Ethanol | Ethanol | | | | | | |
| Uptake | Uptake | UAC | Creatinine | EtG | EtG-100 | EtS | EtS-100 |
| [h:min] | [min] | [g %] | [mg/dL] | [mg/L] | [mg/L] | [mg/L] | [mg/L] |
| 0 | 0 | 0.003 | 170.5 | 1.3 | 0.76 | 0.16 | 0.09 |
| 1:00 | 60 | 0.022 | 77.4 | 5.3 | 6.85 | 2.46 | 3.18 |
| 2:06 | 126 | 0.078 | 17.7 | 12.9 | 72.88 | 4.32 | 24.42 |
| 2:46 | 166 | 0.082 | 14.3 | 7.9 | 55.24 | 6.30 | 44.05 |
| 8:00 | 480 | 0.046 | 71.6 | 49.9 | 69.69 | 23.66 | 33.05 |
| 11:20 | 680 | 0.003 | 72.4 | 30.1 | 41.57 | 15.54 | 21.47 |
| 13:20 | 800 | 0 | 70.1 | 22 | 31.38 | 3.61 | 5.15 |
| 14:50 | 890 | 0 | 144.4 | 26 | 18.01 | 4.78 | 3.31 |
| 16:40 | 1000 | 0 | 112.2 | 11.8 | 10.52 | 2.46 | 2.20 |
| 19:10 | 1150 | 0 | 153.4 | 9.1 | 5.93 | 1.20 | 0.78 |
| 21:30 | 1290 | 0 | 116.6 | 4.5 | 3.86 | 0.83 | 0.71 |
| 26:30 | 1590 | 0 | 112.9 | 1.6 | 1.42 | 0.48 | 0.42 |
| 32:00 | 1920 | 0 | 104.4 | 1.1 | 1.05 | 0.40 | 0.38 |
| 36:00 | 2160 | 0 | 124.6 | 0.5 | 0.40 | 0.22 | 0.18 |

Biological tests can, in addition to self reports, provide clinicians, forensic toxicologists, judges, counselors and program evaluators etc. complementary information. Other roles of biomarkers in alcoholism treatment have been recently discussed by Allen and Litten (2001). These include
serving as outcome variables in treatment efficacy studies,
early identification of relapse in patients in abstinence-oriented interventions, and
serving as a basis for feedback to enhance patient motivation for change.

Granted their ability to monitor heavy drinking use over long periods of time, FAEE, EtS and EtG hair levels may play an important and unique role in both clinical practice and research.

If the question of recent alcohol consumption has to be answered in a binary way (yes/no), like for the question of lapses, the use of EtG or EtS in urine can be suggested as a first choice test. PEth would cover and intermediate time span, comparable to CDT, but with a good chance of doing so with a higher sensitivity and specificity.

An algorithm as suggested by the data on ethanol, FAEE's, EtG, EtS and PEth for the assessment of alcohol intake could be as shown below:

| | Time frame | | | |
|---|---|---|---|---|
| | <1 day | 1-5 days | 2-3 weeks | Months |
| Suggested | Ethanol, | EtG in urine | PEth in | EtG in hair |
| direct markers | EtS in serum | EtS in urine | whole | FAEEs in hair |
| | EtG in serum | | blood | EtS in hair |
| | and urine, | | | |
| | FAEEs in | | | |
| | serum | | | |

Applications for the use of EtS can be such as
screening
motivational feedback
to improve knowledge on drinking patterns
differentiate moderate/social drinking from problematic/harmful drinking
differential diagnosis (e.g. elevated transaminases)
evaluate treatment programs and drug trials
elucidate the role of neuropsychological impairment following alcoholisation (i.e. hangover state) which plays a major role in accidents (Vehicle, but also workplace),
   disclose recent drinking in social drinkers in risky situations (driving, workplaces, pregnancy (FAS))
monitoring
   after liver transplantation and
   methadone maintenance patients (substituted opiate addicts) with hepatitis C and comorbid risky alcohol use,
   all other medical/psychological states, where monitoring alcohol use is required (underage drinking).

These markers may soon come to have important implications in fields of public health and public safety as a more objective method for documenting ethanol exposure among trauma patients, vehicle crash reconstruction, and other forms of alcohol-impaired driver monitoring.

The complementary use of this marker together with other biological state markers and self reports is expected to lead to significant improvement in treatment outcome, therapy effectiveness, and health, social and socio-economic benefits that will be hard to overestimate.

The invention has been described with respect to several particular examples and embodiments. However, the foregoing examples and descriptions are not intended to limit the invention to the exemplified embodiments. The skilled artisan should recognize that variations can be made within the scope and spirit of the invention as described in the foregoing specification. The invention encompasses all alternatives, modifications, and equivalents that may be included within the true scope and spirit of the invention as defined in the summary of the invention.

### REFERENCES

Aderjan, R.; Babel, B.; Briellmann, T.; Daldrup, T.; Demme, U.; Hallbach, J.; Hartung, M.; Harzer, K.; Herbold, M.; von Meyer, L.; Moeller, M.; Musshoff, F.; Schmitt, G.; Weinmann, W. Anlage zu den Richtlinien der GTFCh zur Qualitätssicherung bei forensisch-toxikologischen Untersuchungen. Anhang A: Anforderung an einzelne Analysenmethoden. Toxichem+Krimtech 2000, 67, 13-16. http.//www.gtfch.org/GTFChneuEngl.htm (accessed May 24, 2004)
Allen J P, Litten R Z (2001) The role of laboratory testing in alcoholism treatment. Journal of Substance Abuse and Treatment 20: 81-85 Beck 0, Helander A (2003) 5-Hydroytryptophol as a marker for recent alcohol intake. Addiction 98 [Suppl] 63-72
Aradottir S, Moller K, Alling C (2004) Phosphatidylethanol formation and degradation in human and rat blood. Alcohol Alcohol 39:8-13
Bernstein J (1982) The role of the lung in ethanol metabolism. Res Commun Chem Pathol Pharmacol 38:43-56
Bernstein J, Martinez B, Escobales N, Santacana G (1983) the pulmonary ethanol metabolizing system (PET) Res Commun Chem Pathol Pharmacol 39:49-67
Bernstein J, Meneses P, Basilio C, Martinez B (1984) Further characterization of the pulmonary ethanol metabolizing system (PET) Res Commun Chem Pathol Pharmacol 46:121-36
Bernstein J, Basilio C, Martinez B (1990) Ethanol sulfation by the pulmonary ethanol metabolizing system (PET) Res Commun Chem Pathol Pharmacol 68: 219-34
Bonte W. Begleitstoffe alkoholischer Getränke: Biogenese, Vorkommen, Pharmakologie, Physiologie und Begutachtung. Lübeck, Schmidt-Römhild 1987, pp 142-143
Bonte, W.; Ruedell, E.; Sprung, R.; Frauenrath, C.; Blanke, E.; Kupilas, G.; Wochnik, J.; Zäh, G. Experimental Investigations concerning the Blood-Analytical Detection of Small Doses of Higher Aliphatic Alcohols in Man. Blutalkohol 1981, 18, 299-411.
Boström, H.; Vestermark, A. Studies on ester sulfates. 7. On the excretion of sulphate conjugates of primary aliphatic alcohols in the urine of rats. Acta Physiol Scand 1960, 48, 88-94.
Carlini E J, Raftogianis R B, Wood T C, Jin F, Zheng W, Rebbeck T R, Weinshilboum R M (2001) Sulfation pharmacogenetics: SULT1A1 and SULT1A2 allele frequencies in Caucasian, Chinese and AfricanAmerican subjects. Pharmacogenetics 11:57-68
Dan L and Laposata M. (1997) Ethyl palmitate and ethyl oleate are the predominant fatty acid ethyl esters in the blood after ethanol ingestion and their synthesis is differentially influenced by the extracellular concentrations of their corresponding fatty acids. Alcohol Clin Exp Res 21; 2, 286-292
Diczfalusy M. A., von Wachenfeldt M., Holmberg I., Alexson S. E. H. (2001) Possible role of long chain fatty acid ethyl esters in organ injury and as short-term markers of ethanol intake in humans In New and upcoming markers of alcohol consumption. Wurst F. M. (ed.) Darmstadt, Steinkopff-Springer Verlag, 17-34.
Gilg T, Soyka M (1997) Wertigkeit biologischer Marker für Alkoholabusus und Alkoholismus. Nervenheilkunde 16:362-371.
Helander, A.; Beck, O. Mass Spectrometric Identification of Ethyl Sulfate as an Ethanol Metabolite in Humans. Clin Chem 2004, 5, 936-937.
Helander A (2003) Biological markers in alcoholism. J Neural Transm [Suppl]66:15-32.
Laposata, M. (1999) Assessment of ethanol intake—current tests and new assays on the horizon. Am J Clin Pathol 112: 443450
Manautou J E, Carlson G P (1992) Comparison of pulmonary and hepatic glucuronidation and sulphation of ethanol in rat and rabbit in vitro. Xenobiotica 22: 1309-1319
Varga A Moller K, Hanson P Aradottir S, Alling C (2001) Phosphatidylethanol; clinical significance and biochemical basis. In New and upcoming markers of alcohol consumption. Wurst F. M. (ed.) Darmstadt, Steinkopff-Springer Verlag, pp 75-92
Vestermark, A.; Boström, H. Studies on ester sulfates. V. On the enzymatic formation of ester sulfates of primary aliphatic alcohols. Exp Cell Res 1959, 18,174-177.
Weinmann W, Schaefer P, Thierauf A, Schreiber A, Wurst F M. Confirmatory Analysis of Ethylglucuronide in Urine by Liquid-chromatography/Electrospray-Tandem-Mass-Spectrometry. J Americ Soc Mass Spectrom 2004, 15,188-193
WHO Global Status Report On Alcohol (WHO/HSC/SAB/99.11) (1999) http://www.who.int/substance abuse/pubs_alcohol.htm (accessed, Sep. 10, 2001)
Wurst F M, Kempter, C, Seidl S, Alt A (1999a) Ethyl glucuronide—a marker of alcohol consumption and a relapse marker with clinical and forensic implications. Alcohol Alcohol 34:71-77.
Wurst F M, Schüttler R, Kempter C, Seidl S, Gilg T, Jachau K, Alt A (1999b) Can ethyl glucuronide be determined in postmortem body fluids and tissues? Alcohol Alcohol 34: 260-261.
Wurst F M, Kempter C, Metzger J, Seidl S, Alt A (2000a) Ethyl glucuronide—a marker of recent alcohol consumption with clinical and forensic implications. Alcohol 20: 111-116.
Wurst F. M., Metzger J., Jauchau K., Seidl S., Pridzun L., Janda I., Alt A. (2001) The direct ethanol metabolite ethyl glucuronide: A specific marker of recent alcohol consumption. In New and upcoming markers of alcohol consumption. Wurst F. M. (ed.) Darmstadt, Steinkopff-Springer Verlag, pp 62-74
Wurst F. M., Metzger J. W. on behalf of the WHO/ISBRA study on biological state and trait markers of alcohol use and dependence (2002b) The direct ethanol metabolite Ethyl glucuronide is a specific marker of recent alcohol consumption. Alcohol Clin Exp Res 26: 1114-1119
Wurst F M, Vogel R, Jachau K, Varga A, Alling C, Alt A, Skipper G E (2003a) Ethyl glucuronide discloses recent covert alcohol use not detected by standard testing in forensic psychiatric inpatients. Alcoholism Clinical and Experimental Research 27: 471-476
Wurst F M, Skipper G E, Weinmann W (2003b) Ethyl glucuronide—the direct ethanol metabolite on the threshold from science to routine use. Addiction 98 (Suppl 2) 51-61

Wurst F M, Alexson S, Wolfersdorf M, Bechtel G, Forster S, Alling C, Aradottir S, Jachau K, Huber P, Allen J P, Auwärter V, Pragst F (2004) Concentration of fatty acid ethyl esters in hair of alcoholics: Comparison to other biological state markers and self reported ethanol intake. Alcohol Alcohol 39:33-38

US Patent Documents

| | | | |
|---|---|---|---|
| 5747346 | May 5, 1998 | Pullarkat RK et al | 436/94 |
| 5958785 | Sep. 28, 1999 | Pullarkat RK et al. | 436/94 |

World Intellectual Property Organisation

| | | |
|---|---|---|
| WO 03/010539 A1 | Feb. 6, 2003 | Savolainen M et al |

Wurst F. M., Dresen S., Allen J. P., Wiesbeck G., Graf M., Weinmann, W., Ethyl Sulfate: A Direct Ethanol Metabolite Reflecting Recent Alcohol Consumption. Additiction (2005, in press).
Aderjan, R.; Babel, B.; Briellmann, T.; Daldrup, T.; Demme, U.; Hallbach, J.; Hartung, M.; Harzer, K.; Herbold, M.; von Meyer, L.; Moeller, M.; Musshoff, F.; Schmitt, G.; Weinmann, W. Anlage zu den Richtlinien der GTFCh zur Qualitätssicherung bei forensisch-toxikologischen Untersuchungen. Anhang A: Anforderung an einzelne Analysenmethoden [Amendments to the Guidelines of the GTFCh for Quality Assessment for forensic-toxological analyses: Amendment A: Prerequisites for selected methods}. Toxichem+Krimtech 2000, 67, 13-16. http://www.gtfch.orq.index1.html (accessed Apr. 29, 2005)

What is claimed is:

1. A method for detecting and/or quantifying ethanol intake in a human and/or a non-human animal comprising:
obtaining a body sample from said human and/or non-human animal,
determining a presence or level of EtS in said body sample, and
correlating said presence or level of EtS in said body sample to the ethanol intake of said human and/or non-human animal.

2. The method of claim 1, wherein the presence or level of EtS in said body sample is determined via GC/MS, LC/MS, LC/MS-MS, high pressure liquid chromatography (HPLC), magnetic resonance technology (NMR), Densitometry, Spectroscopy, thin layer chromatography (TLC), capillary electophoresis (CE), transdermal application(s) or fluoresence polarisation.

3. The method of claim 1, wherein the presence or level of EtS in said body sample is determined by one or more antibody based (mono- and polyclonal) determination technique.

4. The method of claim 3, wherein said antibody based determination technique is enzyme linked immuno sorbent assay (ELISA), radio immuno assay (RIA) or a fluorescence based assay.

5. The method of claim 1, wherein the ethanol intake is quantified using deuterium labeled EtS or radio labeled EtS.

6. The method of claim 1, wherein EtS is detected via nanotechnogical methods.

7. Screening and/or monitoring the intake of ethanol in a human via the method of claim 1.

8. The method of claim 1, wherein said method is performed 1 to 24 hours, 1 to 7 days, weeks, or months after ethanol intake.

9. The method of claim 1, wherein said method is combined with at least one second method for determining ethanol intake in a human and/or non-human animal.

10. The method of claim 9, wherein said second method is ethyl glucuronide testing, phosphatidylethanol testing or fatty acid ethyl ester testing and wherein said second method is performed prior and/or after said method of claim 1.

11. The method of claim 1, wherein the presence or level of EtS is measured in more than one type of sample.

12. The method of claim 1, wherein the presence or level of EtS is determined indirectly.

13. The method of claim 12, wherein the presence or level of EtS is determined via one or more enzymes that is involved in the formation or degradation of EtS.

14. A method for detecting and/or quantifying ethanol intake in a human and/or a non-human animal comprising:
obtaining a body sample from said human and/or non-human animal,
determining a ratio of at least two ethanol metabolites in said body sample, and
correlating said ratio of said at least two ethanol metabolites in said body sample to the ethanol intake of said human and/or non-human animal.

15. The method of claim 14, wherein said at least two ethanol metabolites are EtS and EtG.

16. The method of claim 1 further comprising
correlating the ethanol intake and/or the level of EtS determined in said body sample to a level of EtS detected in a food, food-like substance, beverage or medication injested by said human and/or non-human animal prior to or at the time of obtaining said body sample.

17. A method for detecting and/or quantifying EtS in a food, food-like substance, beverage or medication comprising:
providing a sample of said food, food-like substance, beverage or medication, and
determining a presence or level of EtS in said sample.
